(19) United States
(12) Patent Application Publication
(10) Pub. No.: US 2026/0083567 A1
(43) Pub. Date: Mar. 26, 2026

(54) JOINT IMPLANT, PREOPERATIVE PLANNING PROCESS AND ARTHROPLASTY METHOD

(71) Applicant: ADVANCED OSTEOTOMY TOOLS - AOT AG, Basel (CH)

(72) Inventor: Daniel BEER, Niederdorf (CH)

(21) Appl. No.: 19/110,282

(22) PCT Filed: Sep. 29, 2023
(86) PCT No.: PCT/EP2023/077055

§ 371 Date: Mar. 10, 2025

(30) Foreign Application Priority Data
Sep. 30, 2022 (CH) 001141/2022

(51) Int. Cl. A61F 2/38; A61B 18/00; A61B 18/20; A61B 34/10; A61B 90/00; A61F 2/30; G06T 7/00

(52) U.S. Cl. CPC A61F 2/389; A61B 18/20; A61B 34/10; A61B 90/37; A61F 2/30749; A61F 2/30771; A61F 2/30942; G06T 7/0012; A61B 2018/00565; A61B 2018/00577; A61B 2018/00642; A61B 2034/104; A61B 2034/107; A61B 2034/108; A61B 2090/363; A61B 2090/3735; A61B 2090/3762; A61F 2002/30004; A61F 2002/30136; A61F 2002/30171; A61F 2002/30322; A61F 2002/30892; A61F 2002/3093; A61F 2002/30948; G06T 2207/10072; G06T 2207/10101; G06T 2207/30008

(57) ABSTRACT

A joint implant to be mounted to a hard tissue of a patient comprises a body portion. The body portion having a tissue face configured to contact a prepared surface of the hard tissue of the patient, when the joint implant is mounted to the hard tissue of the patient. The tissue face of the body portion comprises an anchoring structure configured to anchor the joint implant on the hard tissue of the patient. The body portion is formed to fit the prepared surface of the hard tissue such that the joint implant is held in a predefined target position when mounted to the hard tissue

### TECHNICAL FIELD

The present invention relates to the provision of an at least partially artificial joint and more particular to a joint implant, arthroplasty method and a process to preoperatively planning an arthroplasty surgery.

### BACKGROUND ART

For treating various joint diseases and malfunctioning of joint it is known to provide an artificial joint or parts thereof. Typically, joint implants are surgically mounted or set, wherein often the implants are integrated in or attached to natural bones adjacent to the joint.

For example, today's surgical treatment options for osteoarthritis (OA) in the knee are total knee arthroplasties (TKA) or partial knee arthroplasties (PKA), most part of the latter being unilateral knee arthroplasties (UKA). In this connection, arthritis is an umbrella term describing several conditions that cause inflammation in joints. In some cases, the inflammation can also affect adjacent skin, muscles, and organs. The most common type is OA compared to rheumatoid arthritis and others.

Often, OA is caused by normal wear and tear on your joints and cartilage. Cartilage is the slippery tissue that covers the ends of bones and helps the joints move. Over time, cartilage can deteriorate and may even disappear completely. This results in bone-to-bone contact in joints, causing pain, stiffness, and sometimes swelling. OA can affect any joint in the body. It's most likely to affect the joints of hips, knees, shoulder, hands, ankle and neck. The risk of developing OA increases with age.

Treatment plan for arthrosis, or other types of arthritis may include: Medication such as over-the-counter (OTC) acetaminophen, nonsteroidal anti-inflammatory drugs (NSAIDs); physical therapy, i.e. a therapist teaches the patient to perform exercises to help strengthen and stabilize joints and regain or maintain range of motion; occupational therapy, i.e. a therapist helps develop strategies to adjust work environment or habits to help manage general condition; orthotics such as braces, splints, or shoe inserts that help relieve stress and pressure on damaged joints; joint surgery, i.e. arthroscopic procedures, arthroplasty or arthrodesis to clean, replace, or fuse damaged joints.

Typically, it is tried that less invasive treatments are applied before surgery, as there are very limited revision options for implants. If all therapies fail, the end state may be severe pain or stiffening which may disable walking. Usually, surgical joint treatments are split in three phases:

Early treatment with lavage or debridement which are both arthroscopic surgeries targeting the removal outgrowth, excessive and interfering cartilage/tissue. Thus, these are mitigation procedures, not addressing or treating the root cause of the inflammation.

Repositioning osteotomies, mainly high-tibia osteotomies (HTO) are surgical interventions to correct malposition and realign the axis, preventing overload of joint surfaces and related consequences.

Joint replacement is most common with mono-block implants for total hip (THA), shoulder (TSA) or knee (TKA) arthroplasty or partially such as unilateral knee arthroplasty (UKA). UKA implants may be used bicondylar for a TKA, what has not yet become established as standard procedure. Implants for further, smaller joints are appearing in niche markets.

Today, implant designs and related procedures limit revision options, particularly considering that an aim of any treatment often is to preserve natural structures such as bone and to restore healthy autologous tissue. To achieve this aim, it has been found that a combination of bone or tissue grafting and thin-walled implant designs may be favourable. However, such solutions require techniques to precisely remove as an exact amount of compromised or damaged structures as necessary.

Even though accurate removal of bone is possible today, known procedures for combined tissue grafting and thin-walled implants are often comparably cumbersome and prone for mistakes, compromising the precision especially of micro-structures. state-of-the-art techniques use mechanical cutting tools such as saw blades or milling cutters.

Furthermore, it may be challenging to precisely hold the thin-walled implants at their target positions and orientations by today's possible anchoring options which are more or less limited to linear or circular geometries, such as flat planes and cylinders or pyramids and cones, respectively.

The design of optimized thin-walled implants typically is restricted by a minimal required wall thickness due to edges caused by the intersection of planar cutting planes created by saw blades.

Therefore, there is a need for systems and components allowing improved procedures to replace natural joints or parts thereof and to maintain as much natural hard tissue such as bone or cartilage as possible.

### DISCLOSURE OF THE INVENTION

According to the invention this need is settled by a joint implant as it is defined by the features of independent claim 1, by a kit as it is defined by the features of independent claim 15, by a preoperative planning process as it is defined by the features of independent claim 16, by an arthroplasty method as it is defined by the features of independent claim 23, and by a treatment method as it is defined by the features of independent claim 35. Preferred embodiments are subject of the dependent claims.

In one aspect, the invention is a joint implant to be mounted to a hard tissue of a patient. The joint implant comprises a body portion that has a tissue face configured to contact a prepared surface of the hard tissue of the patient, when the joint implant is mounted to the hard tissue of the patient. The tissue face of the body portion comprises an anchoring structure configured to anchor the joint implant on the hard tissue of the patient. The body portion is formed to fit the prepared surface of the hard tissue such that the joint implant is held in a predefined target position when mounted to the hard tissue.

The term “hard tissue” as used in the context of the invention can relate to a tissue which is mineralized and/or has a firm intercellular matrix. The hard tissues can particularly be bone tissue, cartilage tissue and/or ligament tissue.

The joint implant can be an orthopedic implant embodied to replace a portion or part of a joint of a human or animal patient. Once implanted it can be referred to as partial or full artificial joint. The joint can be any joint of the human or animal being such as a shoulder or glenohumeral joint, an elbow or cubital joint, a hip joint, or a finger joint. In a particular advantageous embodiment, the joint implant is an artificial portion of a knee joint, i.e. a total knee arthroplasty (TKA) or unilateral knee arthroplasty (UKA).

The anchoring structure may be any geometric or structural design allowing to be integrated in a corresponding structure of the prepared surface of the hard tissue, such as pins or slats of any shape and depth profile.

The joint implant according to the invention allows for being efficiently held on the hard tissue. More specifically, by forming the body portion to fit to the prepared surface it can clip-like be connected to the hard tissue such as a cap or helmet. Thereby, the anchoring structure can allow to increase a surface to contact bone tissue for osseo-integration or healing into the hard tissue. Further, it allows to precisely define the position, i.e., location and orientation, of the joint implant on the hard tissue and, particularly, its prepared surface.

The joint implant can be a thin-walled joint replacement implant with minimal wall thickness. It may be produced patient-specifically based on pre-operative planning data or it can be an of the shelf implant, e.g., provided in confection sizes.

Preferably, the body portion is formed to slide-fit the prepared surface of the hard tissue, i.e., enabling a precise sliding seat on the prepared surface of the hard tissue. The slide-fit may particularly involve frictional, spring and/or clamping forces. Such slide-fit allows for an efficient and convenient mounting of the joint implant to the hard tissue.

Preferably, the anchoring structure of the tissue face of the body portion comprises at least one anti-pivot protrusion having a rotationally asymmetric cross-section. By such asymmetric anti-pivot protrusion, the implant can efficiently and precisely be oriented and located relative to the hard tissue.

In one particularly advantageous embodiment, the at least one anti-pivot protrusion of the anchoring structure of the tissue face of the body portion comprises a plurality of wave-like elevations. Such waves allow to stabilize the implant in plural directions. Thus, compared to, e.g., linear elevations blocking relative one dimensional translational movements or curved elevations blocking relative one dimensional rotational movements, wave-like elevations allow for blocking multi-dimensional rotational and translational movements by means of one geometrical structure. Thus, a particularly efficient fitting of the joint implant to the prepared surface of the hard tissue is possible. Also, such elevations allow for a convenient mounting of the joint implant to the hard tissue and, at the same time, to precisely position the joint implant on the hard tissue.

In another embodiment, the at least one anti-pivot protrusion of the anchoring structure of the tissue face of the body portion comprises a post having an angular or spline polygonal or star-like cross-section. Such post allows for securely connect the joint implant to the hard tissue in order to prevent unintended rotation of the joint implant relative to the hard tissue.

Preferably, the anchoring structure of the tissue face of the body portion comprises at least one form-fit protrusion configured to form-fit a form-fit cavity of the prepared surface of the hard tissue. Such form-fit protrusion allows for providing a comparably solid and safe connection of the joint implant to the hard tissue. In particular, the joint implant can be clipped or snapped on or in the hard tissue or its prepared surface. In particular, the form-fit protrusion interacting with the form-fit cavity allows to block a movement of the joint implant relative to the hard tissue in a predefined direction such as, particularly, a direction away from the hard tissue. For example, the joint implant having such form-fit protrusions can be mounted to the hard tissue in a push button like fashion.

By means of the form-fit protrusion, a specifically beneficial, efficient and stable mounting of the joint implant to the hard tissue can be achieved. Like this, a precise and efficient healing or growing in of the implant into the hard tissue can be achieved at a well-defined position.

Thereby, the at least one form-fit protrusion preferably has a cross-section increasing in a direction intended to be directed towards the prepared surface of the hard tissue. Such protrusion with increasing cross-section allows to provide a comparably easy manufacturable for-fit structure. The form-fit protrusion can be a straight or wave-like bar extending from the tissue face, wherein a cross section increases. For example, the cross-section of such bar can be trapezoid or similar. Also, the form-fit protrusion can be up-side down pyramid-shaped or frustoconical, whereas the planes of the pyramid may be flat, concave or convex and the envelope of the cone can be circular or ellipsoidal.

Preferably, the joint implant is configured to snap-fit the prepared surface of the hard tissue. Thereby, the snap-fit may combine slide-fitting and form-fitting. Such snap-fit configuration allows for a convenient and stable mounting of the joint implant to the hard tissue and to achieve an appropriate holding of the joint implant to the hard tissue.

Preferably, the body portion is made of a biocompatible metal or a metal alloy which may be modified or enhanced with further material or substance to facilitate or promote hard-tissue—or osseointegration. Such body portions are comparably robust and reliable. Also, such materials may be biocompatible such that they are suitable for being implanted.

Preferably, the joint implant comprises a shell portion, wherein the body portion has an exposure face to be exposed towards a part of the joint other than the hard tissue to which the joint implant is to be mounted, and wherein the shell portion is secured to the body portion such that the exposure face of the body portion is covered by the shell portion. The exposure face can be more or less opposite to the tissue face. The shell portion allows for providing different properties or characteristics at hard tissue and exposure faces.

Thereby, the shell portion preferably is made of a cross-linked plastic and, more preferably, the cross-linked plastic is a polyethylene eventually provided with an antioxidant such as vitamin C. Such plastic shell portions allow for providing a comparably soft but still robust design. For example, the shell can be provided to be in contact with another joint implant and to receive the latter.

In a preferred embodiment, the joint implant is configured to be mounted to a knee end of a tibia. In another preferred embodiment, the tissue face is formed to surround a knee end of a femur. Such joint implants allow for providing artificial knees or part thereof. The same may apply to other joints, such as for femur and pelvic in the hip or for the humerus and glenoid in the shoulder, respectively.

In another aspect, the invention is a joint implant kit. The joint implant kit comprises a first joint implant as described above and a second joint implant having a shell portion as described above. The first joint implant comprises a first body portion having a tissue face configured to contact a prepared surface of a first hard tissue of a joint of a patient when the first joint implant is mounted to the first hard tissue of the joint of the patient. The second joint implant comprises a second body portion having a tissue face configured to contact a prepared surface of a second hard tissue of the joint of the patient when the second joint implant is mounted to the second hard tissue of the joint of the patient. The second body portion of the second joint implant has an exposure face to be exposed towards the first joint implant, when the first joint implant is mounted to the first hard tissue of the joint of the patient and the second joint implant is mounted to the second hard tissue of the joint of the patient. The second joint implant has a shell portion secured to the second body portion such that the exposure face of the second body portion is covered by the shell portion.

The joint implant kit according to the invention allows for achieving the effects and benefits of the joint implant and its preferred embodiments described above in an application where plural parts of a natural joint are replaced.

In a further aspect, the invention is a preoperative planning process comprising the steps of: (i) obtaining tissue face data representing the geometry of a tissue face of a body portion of a joint implant as described above; (ii) obtaining image data of an initial situation of a hard tissue of a joint of a patient; (iii) defining a target position of the joint implant on an image of the hard tissue generated by the image data; and (iv) computing a cut geometry using the image and the defined target position to prepare a surface of the hard tissue, wherein the cut geometry is adapted to generate a prepared surface of the hard tissue corresponding to the tissue face of the body portion of the joint implant.

The image obtained may particularly be a medical image. It can depict the initial situation by means of a cross-sectional view of the tissues of the joint.

The prepared surface may have a general form suiting the general form of the tissue face of the joint implant. It may further be provided with a structure mating the anchoring structure of the tissue face of the body portion.

The preoperative planning process can be computer implemented. It can be embodied by means of computer program executed on a computer system.

The preoperative planning process allows for accurately planning the surgical intervention necessary to apply the joint implant to the patient. In particular, it allows to plan the exact surgical intervention to perform on the hard tissue in order to exactly mount the joint implant to the hard tissue. Moreover, it allows for providing an appropriate structure to the hard tissue suitable to accurately receive the anchoring structure.

Preferably, the cut geometry is adapted to remove a volume of hard tissue of the joint up to a target hard tissue of the joint covered by the volume of hard tissue. By exactly removing volume of hard tissue and keeping as much other hard tissue as possible, it can be achieved that comparably few hard tissue has to be cut away such that comparably lot of natural hard tissue can be kept or maintained. Nevertheless, by removing the volume of hard tissue better suitable or better provided tissue of the hard tissue can be accessed, which allows for a more efficient and better healing.

In an embodiment, the target hard tissue is calcified layer hard tissue located between the cortical hard tissue and subchondral bone tissue. Such procedure allows for replacing natural cartilage tissue.

In another embodiment, the target hard tissue is cancellous bone tissue. Anchoring in such cancellous bone tissue allows for a particular efficient healing, particularly when laser ablation is applied which allows for maintaining or only little impairing the natural structure of the cancellous bone tissue.

Preferably, the image data of the initial situation of the hard tissue is obtained pre-operatively by computer tomography (CT) or magnetic resonance imaging (MRI). CT allows for providing precise three-dimensional information about the hard tissue including focal defects thereof. Thus, like this accurate planning of the surgical intervention can be embodied.

Preferably, the preoperative planning process comprises a step of forming the joint implant based on the image data of the initial situation and, advantageously, based on the defined target position. Such forming step allows for designing and providing a patient specific implant (PSI).

Alternatively, the preoperative planning process preferably comprises a step of selecting the joint implant from a plurality of ready-for-use joint implants based on the image data of the initial situation. Such selection allows for providing an implant tailored to the needs of the patient.

In another further aspect, the invention is an arthroplasty method comprising the steps of: (a) obtaining access to a hard tissue of a joint of a patient; (b) preparing a surface of the hard tissue of the joint by providing a laser beam to the hard tissue and thereby ablating a volume of hard tissue up to a target hard tissue covered by the volume of hard tissue, and by providing the laser beam to the target hard tissue thereby generating a structure in the target hard tissue mating an anchoring structure of a tissue face of a body portion of a joint implant as described above; and (c) fitting the body portion of the joint implant to the prepared surface of the hard tissue such that the joint implant is held in a predefined target position on the hard tissue.

Using a laser for removing the volume of the hard tissue allows for an efficient and precise generation of the prepared surface. Also, such laser cutting can be performed in a fully automatic manner. Moreover, laser ablation does not or to a small extent affect hard tissue neighboring the cut such that the structure of the remaining hard tissue can more or less stay as is, i.e., having its natural structure.

More specifically, robot-assisted cold laser ablation can be used in such joint surgery. This allows for hollowing out of specific three-dimensional geometries, creating space for, e.g., bone or tissue grafts, or shaping or morphing of surfaces to create connection geometries for the joint implants in a sophisticated manner.

Based on the features and benefits of laser ablation thin-walled implant designs, reduced to bear mechanical load in lightweight construction with reinforcement ribs and structures for securing translational and rotational forces are feasible. The three-dimensional freeform surface of the implant can be mapped one-to-one in the target hard tissue, creating a highly precise implant bed for a well-fitting slide-fit or form-fit of the implant to the hard tissue.

The unique combination of robot-assisted cold laser ablation and specifically adapted thin-walled implant designs offer new treatment options with maximum hard tissue savings and possibilities for regeneration of bone/tissue, e.g. supported by grafting or other pharmacological or biomedical therapies.

In one preferred embodiment, the target hard tissue is calcified layer hard tissue located between the cortical hard tissue and cancellous hard tissue. By minimally ablating the hard tissue right up to the calcified layer, an efficient replacement of a cartilage is possible. In particular, to a wide extent the natural hard tissue structure can be upheld.

In another preferred embodiment, the target hard tissue is cancellous bone tissue. By anchoring the implant into the cancellous bone tissue an efficient healing can be achieved.

Preferably, the arthroplasty method comprises the steps of analyzing the hard tissue ablated by the laser beam, generating tissue data relating to the analyzed hard tissue and using generated tissue date when providing the laser beam to the hard tissue to ablate the volume of cortical hard tissue to determine when the target hard tissue is reached. By involving tissue data in the provision of the laser beam it can be achieved that the type and structure of the tissue ablated in fact can be considered. In particular, the laser beam can be adjusted in accordance with the tissue actually ablated. Moreover, once the target hard tissue is reached, provision of the laser beam at the same spot can efficiently be stopped. Like this, it can be achieved that a comparably small volume of hard tissue is removed.

Thereby, analyzing the hard tissue and generating the tissue data preferably is performed in real-time. Like this a very efficient prevention of removing target hard tissue can be achieved. Advantageously, the analysis and tissue data generation is performed inter pulse such that pulse by pulse it is determined if the ablation is sufficient or not.

Furthermore, by analyzing the hard tissue diseased, degenerated or dead tissue can be identified and removed. For example, such analysis allows for detecting inflamed or cancerous tissue which can be removed. Like this, an efficient treatment of the joint is possible.

The hard tissue preferably is analyzed by means of laser induced breakdown spectroscopy (LIBS). LIBS allows for a fast, accurate and reliable analysis of the tissue ablated. Thereby, LIBS can determine the elements contained in the plasma plume generated when ablating the hard tissue. Or it can analyze the tissue by providing a separate analysis laser beam to the hard tissue, advantageously, inter pulse.

Preferably, the arthroplasty method comprises the steps of obtaining an image of an initial situation of the hard tissue; defining the target position of the joint implant on the image of the hard tissue; and computing a cut geometry using the image and the defined target position for providing the laser beam to the hard tissue to ablate a volume of hard tissue. The image can particularly be a digital image, e.g., obtained by CT. Defining the target position on the image advantageously is performed digitally, e.g., by means of a computer or the like. Like this, a practitioner can efficiently process the case assessment and planning.

The image of the initial situation of the hard tissue can be obtained in any suitable way. However, as mentioned, it preferably is obtained by CT. Such imaging allows for generating a three-dimensional precise representation of the hard tissue or boundary surfaces. Also, it allows for digitally providing the image which may essentially ease the further processing.

Additionally or alternatively, the arthroplasty method comprises the steps of obtaining a further image of a current situation of the hard tissue and controlling the provision of the laser beam to the target hard tissue in accordance with the further image. Such control of the laser beam provision allows for accurately reflecting the most current situation and status of the hard tissue on the fly or in real-time.

Thereby, the further image of the current situation of the hard tissue preferably is obtained by optical coherence tomography or by means of a navigated fiducial pointer. Like this, the further image can efficiently be obtained during surgery, i.e., intra-operatively.

The image or further image can be a medical image. It further may depict one or plural cross-sections of the tissues of the joint. Further, before or when computing the cut geometry, the image may be further processed, e.g., in a multimodal image model.

Preferably, the arthroplasty method comprises a step of forming the joint implant based on the image of the initial situation and, advantageously, based on the defined target position. Such forming step allows for designing and providing a PSI.

Thereby, the joint implant, a focal defect implant or graft may be formed intra operatively such that it can be adapted on the current situation of the hard tissue. Particularly, in embodiments where the hard tissue is ablated in accordance with real-time analysis of the hard tissue and generation of the tissue data, advantageously by means of LIBS or real-time OCT, a particularly efficient provision of the PSI can be achieved. For example, in cases where focal defects are detected and ablated by means of LIBS and where it cannot be foreseen with appropriate effort how the end situation of the hard tissue will be, such patient specific joint implant forming in run-time allows for embodying an exactly fitting joint implant and, eventually, preventing any correcting measures such as grafting at the hard tissue.

Processes to form the joint implant, a focal defect implant or graft as PSI may involve additive manufacturing such as 3D-printing.

Alternatively, the arthroplasty method preferably comprises a step of selecting the joint implant from a plurality of ready-for-use joint implants based on the image of the initial situation. Such selection allows for providing an implant tailored to the needs of the patient.

In still another further aspect, the invention is a treatment method for treating a joint of a patient comprising the steps of: obtaining an image of the joint; defining undesired hard tissue of the joint on the image of the joint; obtaining access to the joint; and removing the undesired hard tissue by providing a laser beam and thereby ablating the undesired hard tissue of the joint. The image of the joint can be an image of the initial situation of the joint, e.g., obtained pre-operatively and/or a current or actual image of the joint, e.g., obtained intra-operatively. Techniques for obtaining the image(s) may involve CT, OCT or navigation of a fiducial pointer.

The undesired tissue can particularly be diseased tissue, degenerated tissue, dead tissue, inflamed tissue and/or cancerous tissue. Some of these undesired tissues may be involved in focal defects. Thus, such removal of the undesired tissue allows to treat focal defects. Such focal defects often are the source of joint disorders such as arthrosis or osteoarthritis. Thus, by removing the undesired tissue, the joint can efficiently be treated and healed. Moreover, removing such focal defects can be crucial for acceptance and healing joint implants set to the bone.

By the treatment method according to the invention and its preferred embodiments describe below, effects and benefits of the invention and preferred embodiments thereof can be achieved.

The undesired hard tissue can be removed by a robot-assisted cold laser ablation device.

Obtaining access to the joint, e.g., to a targeted implant bed of the joint, for ablating the tissue can be performed from the joint gap through or to the cartilage through or to the calcified layer, through or to the subchondral bone and beyond into the cancellous bone, if required. Particularly, subchondral bone tissue can be accessed like this and inflamed or other impaired tissue can be removed. Alternatively, access to the subchondral bone tissue can be provided from a mid-section of a longbone bottom-up through the cancellous bone, eventually through the subchondral bone to the calcified layer and further into the cartilage, if required. The cutting strategy and sequence may consist of a series of pulses in the following order aiming beam / visualization (optional); ablation laser; LIBS detection/analysis of the response; and depth measurement by OCT or sound detection and analysis of response for depth/progress control. In parallel and continuously there may be a co-axial video recording and illumination of the surgical site and the focal point of the laser. The access and undesired hard tissue can be ablated by slices of surface, layer by layer, under full control of visual imaging. As described below LIBS can analyze the type of tissue and give feedback if favorable or undesired tissue is ablated—supporting the decision to continue ablation or stop. Depth measurement by OCT or sound detection may be a further technique to support the decision to continue ablation or stop, based on pre-operative planning and predefined depth profile.

Tissues ablated in accordance with the invention may involve cartilage, calcified layer, subchondral and cancellous or cortical bone. Cartilage typically is comparably soft, less dense, and has high liquid content: this allows a fast laser cutting progress and a smooth and uniform flat surface, off focus, distributing the energy over larger surface.

Clear visible removal of cell layer by cell layer is possible, wherein color changing by depth from pure white to yellowish with increasing calcium content can be involved. Calcified layer typically is comparably thin, dense and hard. It can be easily ablated by any energy setting. It is a clear visible yellowish layer with reddish subchondral bone shining through. Identification of the calcified layer can be supported by LIBS detecting the calcium content and stopping by a predefined threshold before reaching the layer. Depth can be controlled relative to pre-op planning.

Subchondral hard tissue typically is cancellous bone, i.e., well perfused but no blood oozing out which may compromise ablation or laser detection. It allows clear visible ablation of cavity and detection of different colored cells. Ablation may be supported by LIBS detecting various cell types and stopping at absence of predefined (compromised) cell types. Depth control may further support to carve out a pre-planned segmented volume.

Preferably, defining the undesired hard tissue comprises the steps of: determining an alignment of the anatomy of the joint to balance the joint; and computing a cut geometry using the image and the determined alignment of the joint. Thereby, removing the undesired hard tissue comprises ablating hard tissue of the joint in accordance with the cut geometry. Like this, the joint can be balanced with respect to the tension of ligaments and/or the load to cartilage.

Preferably, defining the undesired hard tissue of the joint comprises: analyzing the hard tissue ablated by the laser beam; and generating tissue data relating to the analyzed hard tissue, wherein the generated tissue date is used when providing the laser beam ablate the hard tissue.

Thereby, analyzing the hard tissue and generating the tissue data preferably is performed in real-time.

The hard tissue preferably is analyzed by means of LIBS. LIBS allows for accurately and quickly identifying the undesired tissue.

Preferably, defining undesired hard tissue of the joint on the image of the joint comprises identifying degenerated, diseased or dead hard tissue. The degenerated, diseased or dead hard tissue can be a focal defect of the cartilage or defective subchondral hard tissue. Particularly, by involving LIBS the identified degenerated, diseased or dead hard tissue can efficiently and accurately be removed.

Preferably, the image of the joint is obtained by CT, MRI, OCT, or the like.

Preferably, the treatment method comprising a step of grafting the hard tissue at a section where the undesired hard tissue is ablated. Such grafting allows for levelling or treating the hard tissue after removing the undesired tissue. This may allow for the remaining hard tissue to maintain its stability or regenerate which may be particularly important when an implant is set.

Preferably, the treatment method comprises a step of fitting a body portion of a joint implant as described above to the prepared surface of the hard tissue such that the joint implant is held in a predefined target position on the hard tissue.

Thereby, the treatment method preferably comprises as step of forming the joint implant based on the image of the initial situation or a step of selecting the joint implant from a plurality of ready-for-use joint implants based on the image of the initial situation.

### BRIEF DESCRIPTION OF THE DRAWINGS

The invention is described in more detail herein below by way of exemplary embodiments and with reference to the attached drawings, in which:

FIG. 1 shows a perspective view of a femoral knee implant as first embodiment of a joint implant according to the invention of a first embodiment of a joint implant kit according to the invention;

FIG. 2 shows a perspective view of a knee end of a femur prepared in accordance with an embodiment of an arthroplasty method according to the invention;

FIG. 3 shows a perspective view of a first tibial knee implant as second embodiment of a joint implant according to the invention of the joint implant kit of FIG. 1;

FIG. 4 shows a perspective view of a second tibial knee implant as third embodiment of a joint implant according to the invention of the joint implant kit of FIG. 1; and

FIG. 5 shows a perspective view of a knee end of a tibia prepared in accordance with the arthroplasty method according to the invention.

### Description of Embodiments

In the following description certain terms are used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented (rotated 90 degrees or at other orientations), and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

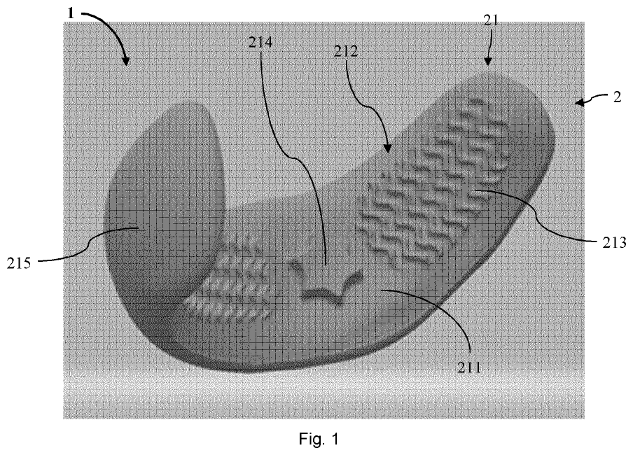

FIG. 1 shows a femoral knee implant 2 as first embodiment of a joint implant according to the invention. The femoral knee implant 2 forms part of a joint implant kit 1 according to the invention. It has a metal body portion 21 generally bow-like shaped in order to be suitable for being press-fitted to a femur 4 of a patient. The body portion 21 has a tissue face 211 configured to contact a prepared surface 41 of the femur 4 of the patient and an exposure face 215 opposite to the tissue face 211. The tissue face 211 comprises an anchoring structure 212 configured to anchor the femoral knee implant 2 on the femur 4 of the patient.

The anchoring structure 212 of the tissue face 211 comprises plural anti-pivot protrusions having rotationally asymmetric cross-sections. In particular, said anti-pivot protrusions comprise a plurality of wave-like elevations 213 and a post 214 having a star-like cross-section.

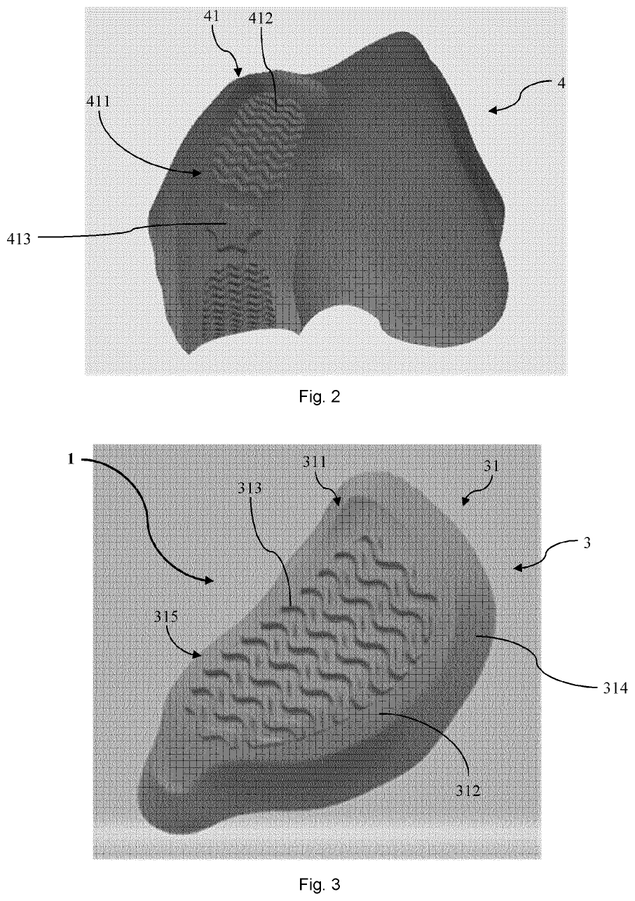

In FIG. 2 a knee end of the femur 4 of the patient is shown, wherein the knee end is provided with a prepared surface 41 to receive the tissue face 211 of the femoral knee implant 2.

Before any intervention, application of the joint implant kit is planned in an embodiment of a preoperative planning process according to the invention. The preoperative planning process comprises a step of OCT scanning the femoral end of the femur 4 and generating tissue face image data representing the geometry of the tissue face 211 of the femoral knee implant 2. A practitioner defines a target position of the femoral knee implant 2 on an image of the femur generated by the image data. This step is performed on a computer system. The computer system calculates a cut geometry using the image and the defined target position to prepare a surface of the femur 4. The cut geometry is adapted to generate the prepared surface 41 of the femur 4 corresponding to the tissue face 211 of the femoral knee implant 2.

The surface of the femur 4 is then treated by an embodiment of an arthroplasty method according to the invention. In this method access to the knee end of the femur of the patient is obtained. Based on the image generated in the preoperative planning process and the defined target position, a cut geometry is computed and the laser beam is provided in accordance with the computed cut geometry. Thereby the surface of the femur 4 of the knee is prepared by providing the laser beam to the femur 4 and ablating a volume of cortical hard tissue up to a cancellous bone tissue of the femur 4 covered by the cortical hard tissue.

For controlling that cartilage and/or bone tissue is as exactly as possible ablated up to the cancellous bone tissue, the tissue ablated is analysed. Therefore, interpulse-wise an analysis laser beam is provided and elements in the plasma plume thereby are analysed in real time by means of laser induced breakdown spectroscopy (LIBS). LIBS generates tissue data relating to the analyzed tissue. And this generated tissue data is used when providing the laser beam to the surface to ablate the volume of hard tissue in order to determine when the cancellous bone tissue is reached. If the generated tissue data show that the ablated hard tissue is degenerated, diseased or dead, ablation is continued until healthy cancellous bone tissue is identified.

Once the healthy cancellous bone tissue is reached, the laser beam is provided to generate a structure 411 in the cancellous bone tissue mating the anchoring structure 212 of the tissue face 211 of the body portion 21. The structure 411 comprises wave-like indentations 412 corresponding to the wave-like elevations 213 of the femoral knee implant 21 and a star-like recess corresponding to the post 214 of the femoral knee implant 2.

After preparation, the body portion 21 of the femoral knee implant 2 is fitted to the prepared surface 41 of the femur 4 such that the femoral knee implant 2 is held in a predefined target position on the femur 4. For achieving this, the body portion 21 of the femoral knee implant 2 is curve-like shaped to fit the prepared surface 41 of the femur 4.

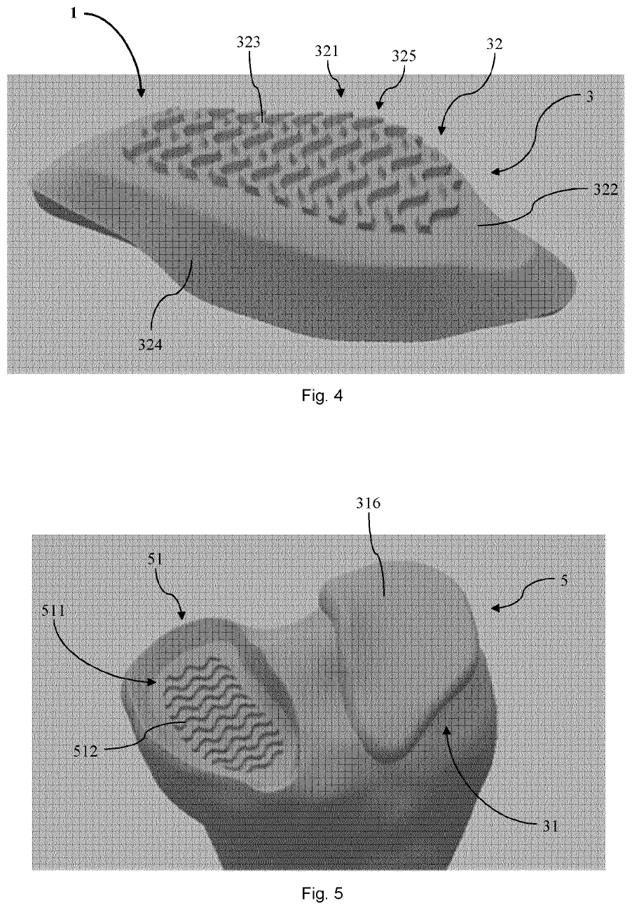

FIG. 3 shows a first tibial knee implant 31 as second embodiment of a joint implant according to the invention of a tibial knee implant set 3 of the joint implant kit 1. In FIG. 4 a second tibial knee implant 32 as third embodiment of a joint implant according to the invention of the tibial knee implant set 3 of the joint implant kit 1 is shown. The first and second tibial knee implants 31, 32 have metal body portions 311, 321 dimensioned to be positioned on the knee end of a tibia 5. The body portions 311, 321 have tissue faces 312, 322 equipped with an anchoring structure 315, 325 having a plurality of parallel wave-like elevations 313, 323 as anti-pivot protrusions. Further, the tibial knee implants 31, 32 have shells 314 324 covering an exposure face 316 opposite to the tissue faces 311, 321.

FIG. 5 shows the knee end of the tibia 5 which is has two prepared surfaces 51 provided by means of the preoperative planning process and the arthroplasty method as described above in connection with the femoral knee implant. Thereby, the prepared surfaces 51 form a structure 511 mating the anchoring structure of the respective tibial implant 31, 32. In particular, the prepared surfaces 51 have cavities equipped with wave-like indentations 512 corresponding to the wave-like elevations 313, 323 of the tibial knee implants 31, 32. In FIG. 5, the first tibial knee implant 31 is fitted into the right-hand prepared surface of the tibia 5 such that an exposure face 315 of the body portion 311 is oriented towards a cavity of the knee.

This description and the accompanying drawings that illustrate aspects and embodiments of the present invention should not be taken as limiting—the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below.

The disclosure also covers all further features shown in the Figs. individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

1. A joint implant to be mounted to a hard tissue of a patient, comprising
a body portion having a tissue face configured to contact a prepared surface of the hard tissue of the patient, when the joint implant is mounted to the hard tissue of the patient;
wherein the tissue face of the body portion comprises an anchoring structure configured to anchor the joint implant on the hard tissue of the patient, and
wherein the body portion is formed to fit the prepared surface of the hard tissue such that the joint implant is held in a predefined target position when mounted to the hard tissue.

2. The joint implant of claim 1, wherein the body portion is formed to slide-fit the prepared surface of the hard tissue.

3. The joint implant of claim 1, wherein the anchoring structure of the tissue face of the body portion comprises at least one anti-pivot protrusion having a rotationally asymmetric cross-section,
wherein the at least one anti-pivot protrusion of the anchoring structure of the tissue face of the body portion preferably comprises
a plurality of wave-like elevations, and/or
a post having an angular, spline polygonal or star-like cross-section.

4.-5. (canceled)

6. The joint implant of claim 1, wherein the anchoring structure of the tissue face of the body portion comprises at least one form-fit protrusion configured to form-fit a form-fit cavity of the prepared surface of the hard tissue,,
wherein the at least one form-fit protrusion preferably has a cross-section increasing in a direction intended to be directed towards the prepared surface of the hard tissue.

7. (canceled)

8. The joint implant of claim 1, configured to snap-fit the prepared surface of the hard tissue.

9. The joint implant of claim 1, claims, wherein the body portion is made of a biocompatible metal or metal alloy, optionally, modified or enhanced with an osseointegration promoter.

10. The joint implant of claim 1, comprising a shell portion wherein the body portion has an exposure face to be exposed towards a part of the joint other than the hard tissue to which the joint implant is to be mounted, and wherein the shell portion is secured to the body portion such that the exposure face of the body portion is covered by the shell portion wherein preferably
the shell portion is made of plastic,
the plastic is a cross-linked polyethylene, optionally, provided with an anti-oxidant such as vitamin C, and/or
the joint implant is configured to be mounted to a knee end of a tibia.

11.-13. (canceled)

14. The joint implant of claim 1, wherein the tissue face is formed to surround a knee end of a femur.

15.-22. (canceled)

23. An arthroplasty method comprising:
obtaining access to a hard tissue of a joint of a patient;
preparing a surface of the hard tissue of the joint by providing a laser beam to the hard tissue and thereby ablating a volume of hard tissue up to a target hard tissue covered by the volume of hard tissue, and by providing the laser beam to the target hard tissue thereby generating a structure in the target hard tissue mating an anchoring structure of a tissue face of a body portion of a joint implant according to claim 1; and
fitting the body portion of the joint implant to the prepared surface of the hard tissue such that the joint implant is held in a predefined target position on the hard tissue.

24. The arthroplasty method of claim 23, wherein the target hard tissue is calcified layer tissue located between the cortical hard tissue and subchondral bone tissue or the target hard tissue is cancellous bone tissue.

25. (canceled)

26. The arthroplasty method of claim 23, comprising: analyzing the hard tissue ablated by the laser beam; generating tissue data relating to the analyzed hard tissue; and using generated tissue data when providing the laser beam to the hard tissue to ablate the volume of cortical hard tissue to determine when the target hard tissue is reached, wherein preferably
analysing the hard tissue and generating the tissue data is performed in real-time, and/or
the hard tissue is analysed by means of laser induced breakdown spectroscopy.

27.-28. (canceled)

29. The arthroplasty method of claim 23, comprising: obtaining an image of an initial situation of the hard tissue; defining the target position of the joint implant on the image of the hard tissue; and computing a cut geometry using the image and the defined target position for providing the laser beam to the hard tissue to ablate the volume of hard tissue,
wherein the image of the initial situation of the hard tissue preferably is obtained by computer tomography.

30. (canceled)

31. The arthroplasty method of claim 23, comprising: obtaining a further image of a current situation of the hard tissue and controlling the provision of the laser beam to the target hard tissue in accordance with the further image,
wherein the further image of the current situation of the hard tissue preferably is obtained by optical coherence tomography or by means of a navigated fiducial pointer.

32. (canceled)

33. The arthroplasty method of claim 23, comprising
forming the joint implant based on the image of the initial situation, or
selecting the joint implant from a plurality of ready-for-use joint implants based on the image of the initial situation.

34. (canceled)

35. A treatment method for treating a joint of a patient comprising:
obtaining an image of the joint;
defining undesired hard tissue of the joint on the image of the joint;
obtaining access to the joint; and
removing the undesired hard tissue by providing a laser beam and thereby ablating the undesired hard tissue of the joint.

36. The treatment method of claim 35, wherein defining the undesired hard tissue comprises determining an alignment of the anatomy of the joint to balance the joint; and
computing a cut geometry using the image and the determined alignment of the joint, wherein removing the undesired hard tissue comprises ablating hard tissue in accordance with the cut geometry.

37. The treatment method of claim 35, wherein defining the undesired hard tissue of the joint comprises: analyzing the hard tissue ablated by the laser beam; and generating tissue data relating to the analyzed hard tissue, wherein the generated tissue date is used when providing the laser beam ablate the hard tissue, wherein preferably
analysing the hard tissue and generating the tissue data is performed in real-time, and/or
the hard tissue is analysed by means of laser induced breakdown spectroscopy.

38.-39. (canceled)

40. The treatment method of claim 35, wherein defining undesired hard tissue of the joint on the image of the joint comprises identifying degenerated, diseased or dead hard tissue.

41. The treatment method of claim 35, wherein the image of the initial situation of the joint is obtained by computer tomography, optical coherence tomography or a navigated fiducial pointer.

42. The treatment method of claim 35, comprising
grafting the hard tissue at a section where the undesired hard tissue is ablated, or
fitting a body portion of a joint implant to the prepared surface of the hard tissue such that the joint implant is held in a predefined target position on the hard tissue,
wherein the treatment method preferably comprises
forming the joint implant based on the image of the initial situation, or
selecting the joint implant from a plurality of ready-for-use joint implants based on the image of the initial situation, and
wherein the joint implant comprises
a body portion having a tissue face configured to contact a prepared surface of the hard tissue of the patient, when the joint implant is mounted to the hard tissue of the patient;
wherein the tissue face of the body portion comprises an anchoring structure configured to anchor the joint implant on the hard tissue of the patient, and
wherein the body portion is formed to fit the prepared surface of the hard tissue such that the joint implant is held in a predefined target position when mounted to the hard tissue.

43.-45. (canceled)
